(19) United States
(12) Patent Application Publication
(10) Pub. No.: US 2017/0245764 A1
(43) Pub. Date: Aug. 31, 2017

(54) DAMAGE DETECTION AND BIO-MONITORING SYSTEM

(71) Applicant: Revision Military S.a.r.L., Luxembourg (LU)

(72) Inventor: STEVE CARKNER, Ottawa (CA)

(73) Assignee: Revision Military S.a.r.L., Luxembourg (LU)

(21) Appl. No.: 15/444,913

(22) Filed: Feb. 28, 2017

Related U.S. Application Data
(60) Provisional application No. 62/301,074, filed on Feb. 29, 2016.

(51) Int. Cl. A61B 5/0205; A61B 5/00; F41H 5/04

(52) U.S. Cl. CPC A61B 5/0205; F41H 5/0478; A61B 5/6805; A61B 5/6803; A61B 5/02438

(57) ABSTRACT

A bio-monitoring system for armor plates. A piezoelectric sensor is used to measure flexing of an armor plate resulting from a wearer's respiration. The resulting measurements are used to determine at least one of a respiration rate and a heart rate of the wearer. Additional bio-monitoring sensors may be used. The system may be used in combination with an armor plate damage detection system.

### RELATED APPLICATIONS

This application claims the benefit under 35 U.S.C. §119(e) to U.S. Provisional Application Ser. No. 62/301,074, entitled “Damage Detection and Bio-Monitoring System,” and filed on Feb. 29, 2016, which is herein incorporated by reference in its entirety.

### FIELD

Embodiments of the present disclosure relate to the field of armor protective plates and accessories often worn by soldiers, police, and others. a method and system that will allow such plates and accessories to provide bio-monitoring functions as a dual-use device. More particularly using wave technology in combination with other electronic systems, to detect, record and communicate a damage level status of an armor plate and accessory, and health status of a user wearing the armor plate and accessory.

### DISCUSSION OF THE RELATED ART

Historically, armor was made of metal or wood and deformed to absorb the impact of a projectile. Damage to armor components, such as plates, either from a projectile, or from inadvertent impact, were visually detectable by the wearer or others. Visual indicators such as cracks, dents and other deformities could be used to indicate weakness in the armor.

Modern armor typically includes several materials that work in combination. Ceramic armor worn by a soldier typically has a layer of ceramic armor plates to shred the projectile, followed by a layer of ultra-high molecular weight plastic, often with fiber reinforcement, which stops the remaining projectile fragments. The armor plates may have a soft backing material that provides comfort to the soldier and room for back-face deformation of the armor plate as the armor plates absorb the projectile energy. The entire armor plate assembly may be encased in a fabric, fiberglass, plastic or other type of protective material to prevent scuffing, minimize edge damage, and provide a uniform look to the final product.

Ceramic is a delicate substance. If a soldier accidentally drops their armor, the ceramic plate may crack, and the armor may become less effective in the area of the crack. The level of damage may not be visually detectable to the wearer as the internal ceramic plate may be covered by multiple layers of material. Therefore, the soldier may unknowingly wear an armor plate that does not provide adequate or expected levels of protection.

One accepted method of plate testing uses an X-ray machine to visually scan the internal features of the plate. This method of inspection is adequate for home-land inspection purposes. However, the process requires specialized equipment and staff and is not accessible at the front lines of a battle-zone.

X-Ray inspection methods also lack the immediacy that is sometimes required at the front-lines of a battle situation where a soldier may have only a few moments to select an armor plate and gear-up for deployment.

X-Ray inspection may also miss changes to the overall plate system as a whole. For example if different layers of the plate have delaminated from each other or have absorbed water or other chemicals, they may be weaker than expected, yet will show no physical damage, such as cracking, when X-Ray inspected.

### SUMMARY

According to one embodiment, a method of measuring respiration rate includes using a sensor on an armor plate to sense a flexing of the armor plate while the armor plate is worn by a user to produce flexing measurements. The method also includes determining at least one of a respiration rate and a heart rate of the user based on at least the flexing measurements.

According to another embodiment, a device includes an armor plate and a sensor positioned on the armor plate to measure flexing of the armor plate. The device also includes a processor configured to determine at least one of a respiration rate and a heart rate of a wearer of the armor plate, the processor using flexing data produced by the sensor.

According to a further embodiment, a device includes an armor plate and a first sensor on the armor plate. The first sensor is configured to measure flexing of the armor plate when the armor plate is worn by a user. The device further includes a temperature sensor on the armor plate, and a perspiration sensor on the armor plate.

### BRIEF DESCRIPTION OF THE FIGURES

For a more complete understanding of the embodiments of the present invention, the objects and advantages thereof, reference is now made to the ensuing descriptions taken in connection with the accompanying drawings briefly described as follows.

FIG. 1a shows a cross sectional view of a composite armor plate, according to an embodiment of the invention;

FIG. 1b illustrates a change in the location of transducer element, to another embodiment being between the ceramic and plastic layers, according to an embodiment of the invention;

FIG. 2 shows a cross-sectional view of the front face of an armor plate, with a transducer or sensor unit located inside the armor plate, according to an embodiment of the invention;

FIG. 3 illustrates a cross-sectional view of a composite armor plate, with a transducer element having a single active area, such as a piezoelectric disk, according to an embodiment of the invention;

FIG. 4 illustrates a cross-sectional view of a composite armor plate, with a transducer element having a separate transmitter and receiver, according to an embodiment of the invention;

FIG. 5a shows the movement of a transverse wave through undamaged material at an initial point in time, according to an embodiment of the invention;

FIG. 5b shows the movement of a transverse wave through a material, at a subsequent point in time relative to the point of time in FIG. 5a, where the wave is reflecting off edges of the material and moving back toward a sensor unit, according to an embodiment of the invention;

FIG. 6a shows the movement of a transverse wave through a damaged material, at an initial point in time, according to an embodiment of the invention;

FIG. 6b shows the movement of a transverse wave through a damaged material, at a subsequent point in time to that in FIG. 6a, where the wave is reflecting off edges of the material and a damaged portion of the material, and where the reflected waves are moving back towards the transducer, according to an embodiment of the invention;

FIG. 7a illustrates circuitry of an armor plate system that connects elements, units and components of the system, according to an embodiment of the invention;

FIG. 7b illustrates an external circuit system for an armor plate system where the transducer and memory element remain on the armor plate, and the remaining components may be configured to complete the circuit from a location other than the armor plate, according to an embodiment of the invention;

FIG. 8 is a process flow diagram of methods of an armor plate system, according to an embodiment of the invention;

FIG. 9 illustrates one embodiment of a damage detection system and bio-monitoring system on a protective accessory;

FIG. 10 shows a front face of a ballistic armor plate with an embedded transducer being worn by a user, according to an embodiment; and

FIG. 11 shows a representative waveform for respiration and heartbeat, according to an embodiment.

### DETAILED DESCRIPTION

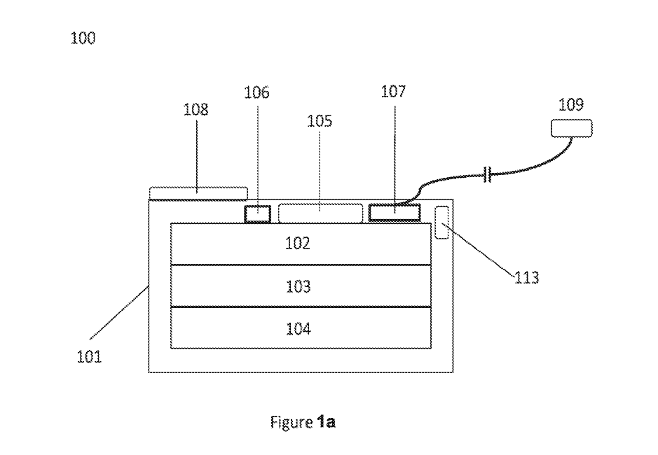

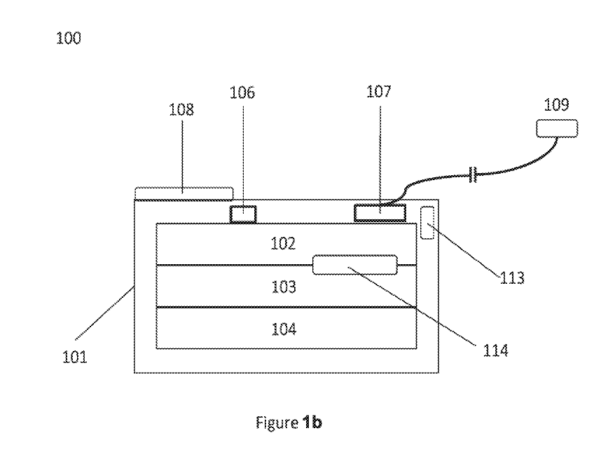

Various embodiments may be understood by referring to FIGS. 1-11, wherein like reference numerals refer to like elements.

FIG. 1a illustrates a cross section of a composite armor plate 100 according to some embodiments. The composite armor plate has three layers. The first layer of the plate may have a layer of ceramic material or other material 102, which functions to break up an incoming projectile when the projectile comes into contact with the ceramic layer. In an embodiment, the second layer may include a ultra-high molecular weight fiber reinforced plastic or other material 103, which functions to shield the first layer from the broken-up projectile fragments. The broken up projectile fragments may deform the plastic layer as a result of the force on contact. In an embodiment, the third layer may be a comfort layer 104, composed of foam, rubber or other material. The comfort layer 104 functions to absorb deformation that may occur in the plastic layer 103, and provides a cushion to the body to reduce trauma. In an embodiment, the ceramic, plastic and comfort layers and/or composite armor are encased in an outer layer 101 such as fabric, plastic, carbon fiber, fiberglass or some other suitable material. In an embodiment, the armor plate is uniform in appearance such that a user is unable to visually inspect the composite armor to look for damage that may have been caused by abuse, accident, dropping, chemical exposure, age, humidity or other causes.

In an embodiment, FIG. 1a shows an armor plate system 100 having a transducer unit 105, a memory unit 106, and a microprocessor unit 107. In an embodiment, the armor plate system has a display unit 108 that allows a user to view the results of a test scan of the armor plate system. In an embodiment, the plate system has a button or switch 109 that can be pressed to activate the test circuit. In an embodiment, the armor plate system has a wireless transmitter unit 113 that can communicate with a remote person or entity. In one embodiment, the results of a test scan are communicated with a remote person or entity.

The composite armor 100 can be configured in a wide variety of ways, and FIGS. 1a and 1b are used strictly for illustrative purposes. In some embodiments, the plates can be made with more or less layers. In other embodiments, the order of the layers and the composition of the layers can be changed while still retaining the features of a composite armor plate system.

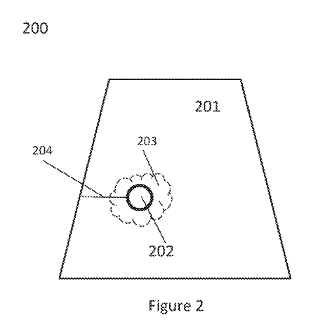

In an embodiment, FIG. 1a illustrates that the system has a single transducer or single sensor unit 105 that is attached to the layer of ceramic material 102 on the outside surface (between ceramic 102 and covering 101). In one embodiment, FIG. 1b shows that the transducer component can be located on the inside surface, between the ceramic 102 and plastic 103 layers, as this further protects the sensor. In some embodiments, the mounting location of the sensor unit on or around the plate or other layers may be changed because of the flexibility of the system. In an embodiment, FIG. 2 illustrates that one factor in determining the location of the sensor may be an important factor in deciding the location of the sensor is the distance 204 from the sensor 202 to the nearest plate edge, as this is the shortest distance for any waveform produced by the sensor to travel.

In an embodiment, FIG. 2 shows a localized cross-sectional area of an armor plate system 200, which is denoted by the cloud-like area 203, to illustrate that the sensor unit 202 can be inside the armor plate 201.

In one embodiment, FIG. 2 shows a front facing perspective of a fully assembled armor plate 200 that can be worn by a soldier, for example on the chest. In other embodiments, the armor plate 200 can be tailored to any body size or shape. For example, the armor plate 201 can be modified in shape and size to fit a variety of body parts and shapes, such as complex curves, thinner areas and attachment points, as influenced by an intended application or environment, the desired protection level, and the user wearing the plate. In another embodiment, the armor systems disclosed herein are not limited to the type of body (human or otherwise) or limited to fit only a single person. In some embodiments, the armor system may be usable with vehicles or other mechanical equipment.

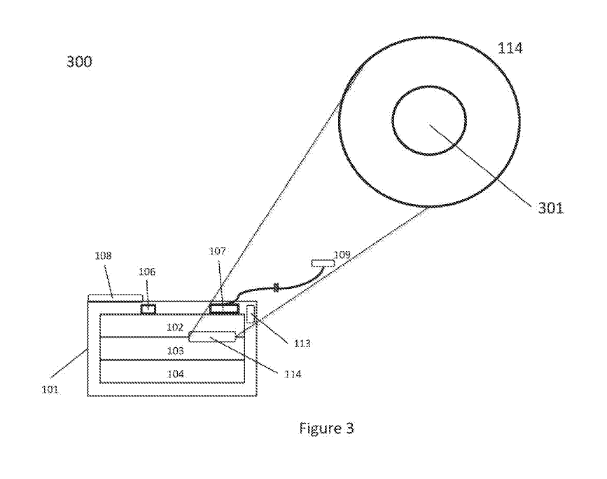

In an embodiment, FIG. 3 shows that the sensor unit 114 may comprise a piezoelectric disk 301. Piezoelectric disks can be durable, light, thin, temperature stable, inexpensive and readily available, although other suitable sensors may be used. In an embodiment, the sensor unit 114 sends a transverse wave into the ceramic material or other suitable material, wherein the sensor unit 114 flexes perpendicular to the surface of the ceramic material, which causes a wave to radiate out from the sensor unit 114 in all directions.

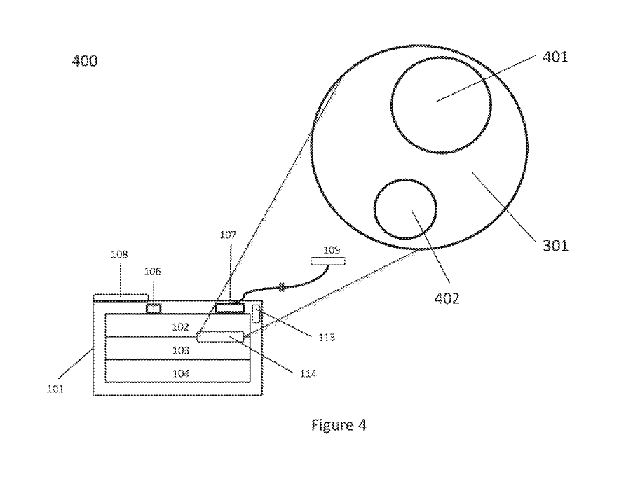

Piezoelectric disks may create electricity when they are flexed. In an embodiment shown in the magnified view of FIG. 4, sensor unit 114 may comprise a piezoelectric disk 301, where the sensor unit 114 is a transducer that is configured to transmit signals via a transmitter component 401 and receive signals via a receiver component 402. The sensor unit 114 produces mechanical energy by physically flexing in response to electricity, and produces electricity when flexed.

Other sensors may be used which produce a surface wave through compression of the ceramic surface. These sensors are typically composed of hundreds of layers of piezoelectric materials and are typically directional in nature, transmitting and receiving energy more strongly in certain directions. In other embodiments, other sensor types can be used, such as magnetic coils, painted-on electro-active coatings, microelectromechanical devices, or any other sensor that can both generate and respond to mechanical energy.

In one embodiment, a single sensor 202,114 is used to both transmit and receive mechanical energy from within the plate, where the injected waveform from the sensor is short enough so that it does not interfere with the received waveform.

The speed at which a mechanical waveform travels in the armor plate is dependent on the materials present. In air, a mechanical waveform (e.g., sound) travels at about 340 m/s (meters per second). The speed of a mechanical waveform in ceramics commonly used in military armor is about 6000 m/s.

In an embodiment, FIG. 2 represents an example, where the distance D 204 is the closest distance, in meters, from the sensor 202 to the edge of the plate. Given the speed of a waveform in the material is V, then the shortest time T for a wave to leave the sensor and return would be:

T=2D/V

In one embodiment a distance D equal to is 0. lm and the velocity V equal to 6000 m/s, would result in wave leaving the sensor and returning in a time T equal to 33μS.

It is known in the art that when a piezoelectric disk is excited, it flexes, but when the signal is taken away, it may continue to vibrate for a short time at its natural resonant frequency—sometimes referred to as self-excitation or ringing of the sensor. Ringing can be damped using mechanical and electrical means. Overdamping the sensor will limit ringing, but may lead to loss of sensitivity. A properly damped sensor may require an additional time of three to five wavelengths of the excitation pulse for the sensor to settle before receiving the returned pulse.

Given the desirability for N wavelengths for ringing to settle, we find the excitation pulse length L may be shorter than:

L=2D/VN

In one embodiment, there is a distance of 0.1 m to the plate edge, a velocity of 6000 m/s and three wavelengths of settling time. This results in an excitation pulse length of 11 μs.

In other embodiments, shorter excitation pulses, or repeated excitation pulses can be used at frequencies much higher than the example shown above. As the frequency increases, there may be more signal loss as the returned waveform may become averaged out by the sensor itself.

The diameter of the sensor 202, 114 may produce the maximum signal when the mechanical waveform length matches the diameter of the sensor. At this point, the mechanical flex of the sensor may be minimized along the edges of and maximized in the center of the sensor (or vice versa as the wave passes). A selected diameter of the sensor therefore may depend on the excitation pulse chosen in combination with the material properties.

In the following equation, d is the sensor diameter, L is the chosen excitation pulse length in seconds and V is the velocity of the wave.

d=LV

In an embodiment, a wave of 11 μs in a material with a mechanical waveform speed of 6000 m/s, the calculated sensor diameter would be 11 μs×6000 m/s, or 6.6 cm. As noted above, the length of the pulse can be made shorter than the calculations show. In an embodiment, for example, if L is 5 μs and V is 6000 m/s, then d will be 3 cm. A 3 cm sensor may still provide a desirable sensitivity, cost and performance.

A possible limiting factor in choosing smaller sensors is related to the frequency of operation. As a sensor gets smaller, the frequency at which it is operated is typically increased. Higher frequencies are generally absorbed by surrounding materials, so the sensitivity may be compromised if the sensor is too small.

With the calculations provided above and bearing in mind the material properties to be measured, an appropriate size sensor and excitation frequency can be chosen for the system.

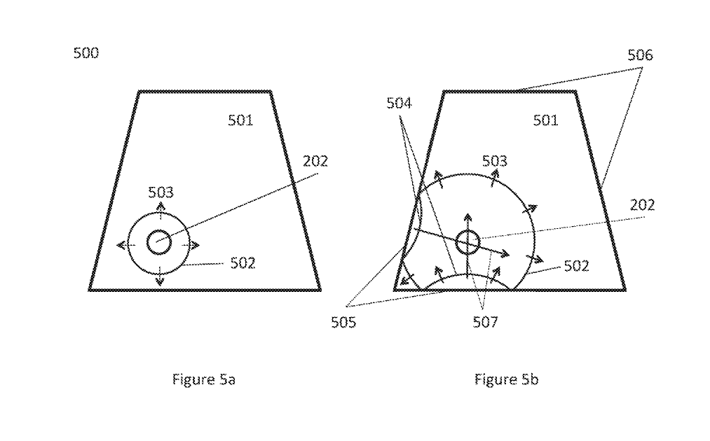

In an embodiment, FIGS. 5a and 5b show an armor plate damage detection system 500 where the sensor unit 202 has been activated and where its action is depicted at two subsequent points in time (FIG. 5a and FIG. 5b). FIG. 5a illustrates that after injecting the mechanical pulse of energy into the armor plate material 501, an initial wave is generated 502 from the sensor unit 202, 114 that radiates away 503 from the sensor 202. FIG. 5b illustrates that at a subsequent point in time, the initial radiating waves 502 are reflected 504 by the edges of the plate 505, 506 and by any other features associated with the mechanical and material properties of the plate. The plate edges 505 closest to the sensor 202 reflect the wave energy first, followed by the other edges 506. In addition, it is well known in the art that energy reflected from one edge can intersect and reflect from another edge (not shown in the figures). As this mechanical energy travels, it crosses 507 and re-crosses (not shown) the sensor unit 202 at further subsequent points in time. The wave and other energies eventually dissipate due to the materials and repeated reflections at the edges.

In an embodiment, when a plate 501 is first manufactured and known to be undamaged, the waveform produced by a known injection pulse 502, 504 can be recorded, for example in the memory component. The representation of the received electrical signal may be stored as a known-good value in a non-volatile memory, and the non-volatile memory may be attached, in some cases permanently, to the armor plate system. The recording of this injection pulse when the plate is undamaged can serve as a baseline that can be used in a processing method in the microprocessor 107 to analyze to future recordings. In an embodiment, at a future point in time, a subsequent injection pulse can be produced and the reflected waveform analyzed with reference to the baseline stored in the memory element.

In an embodiment, the method of creating and storing a baseline waveform improves manufacturing of the system as slight changes in the sensor, the mounting location, processing and plate sizes can be calibrated out of the system.

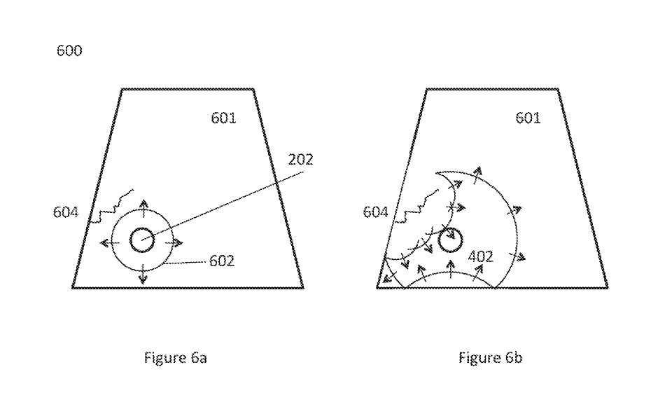

In an embodiment, FIGS. 6a and 6b show two subsequent points in time 600 during a test signal, where a damaged armor plate 601 has a physical defect or crack 604. At a first point in time (FIG. 6a), the sensor unit is activated, generating a waveform 602 that begins to travel away from the sensor unit 202, much like in FIG. 5a. However, before the waveform reaches a particular edge of the material, its path is interrupted by the edges of the damaged portion of the material. In FIG. 6b, when the wave reaches the damaged portion of the plate 604, it reflects off the edges of the damaged material and travels back towards the sensor 202. Unlike in FIG. 5b where the waveform is not interrupted prior to reaching the edges of the material, in FIG. 6b, the reflected waves are returning from the edges of the cracks in the plate and not the edges of the plate. The returning or reflected waveform in FIG. 6b results in a particular waveform being received by the sensor and will be different to that received by the sensor in FIG. 5b.

In other embodiments, the particular waveform may vary and change based on the type and extent of the damage in the armor plate system so that the recorded test signal is a representation of the type and extent of the damage in the armor plate system. In an embodiment, the armor plate is delaminated or is chemically or biologically compromised, resulting in an alteration of the physical integrity of the armor materials, which generates a particular waveform in the signal received by the sensor 202, 114. For example, if the ceramic material has absorbed a chemical from the environment that softens the ceramic material, the velocity of the waveform and the propagation losses may increase. As a result, the test would detect significant shifts in both the timing and amplitude response of the returned waveform. In a similar fashion, delamination of the structure creates irregular reflections between the material layers as the waveform propagates in a three-dimensional fashion through all materials of the plate. It is expected that the soft comfort layer, for example, would absorb the test pulse energy. But if it is delaminated in a region of the plate, that region will not absorb the energy and will result in higher than expected localized reflections. In a more particular embodiment, any other change in the armor plate system that can generate an altered waveform can be recorded by a test signal.

In an embodiment, the difference between the signature waveform and the subsequent tested waveform can be detected, and if the result is outside of the normal variation for that armor plate, as determined through test and analysis of similar systems, the plate would be considered to have failed the test. In particular embodiments, a failure result can be based on a number of statistical analysis functions that can be, but are not limited to, one or more of the following: simple as point-for-point comparison of the waveform, total energy analysis, wave shape detection, difference detection (through subtraction of the waveforms), Fourier analysis, binning, averaging, and time-slicing.

In an embodiment, the magnitude of the difference between the expected signals or signature waveform and the test signal is a factor when analyzing the results or data received from the sensors. In particular embodiments, extensive or significant damage to the plate results in drastic changes to a waveform, while minor damage causes more minor changes to the waveform relative to a significant damage.

In an embodiment, the magnitude of the change or relative difference in waveform can be presented on a normalized scale, such as a display 108, percentage indicator, or an LED indicator with multiple indicator lights. In an embodiment, the results that are processed and communicated on a display give the user a more accurate and reliable indication of the extent and nature of the damage to the plate due to the quantifiable measure and analysis of the test signal. In an embodiment, other analysis methods can be integrated into the test system configured to generate specific information about the state of the armor plate.

In some embodiments, a plate system that produces a result of no significant change in the returned waveform can be interpreted as 100% healthy. In an embodiment, an interpretation is communicated on a 5-LED indicator, where for example all 5 LEDs can be lit when the user initiates a plate test that is 100% healthy. In another embodiment, a plate system digitally communicates an interpreted message with a remote monitor. For example, a message such as “100%”, or another message, can be sent to the monitoring system to indicate the plate is undamaged.

In a further embodiment, if the plate has relatively minor damage as measured by minor changes in the returned waveform, an indication of 80% healthy might be produced, similarly only 4 LEDs might be lit on a health indication display to reflect this relative level of damage.

In a further embodiment, if the plate has major damage, the indicator may drop to 20% or even 0%. In an embodiment, the LED scale and relative damage calculated can be calibrated based on the level of acceptable damage for a particular environment.

In an embodiment, a user, when faced with a variety of plates to choose from for a mission, can choose the plate with the highest state of health. If all of the plates in inventory show some level of damage, embodiments of the system disclosed herein may provide the soldier with the ability to select the least damaged plate for their mission.

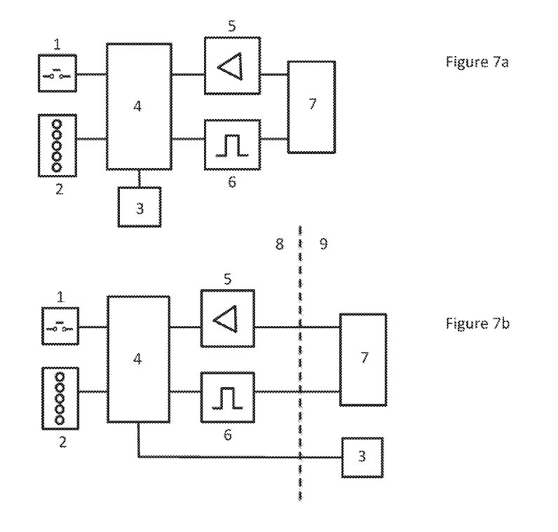

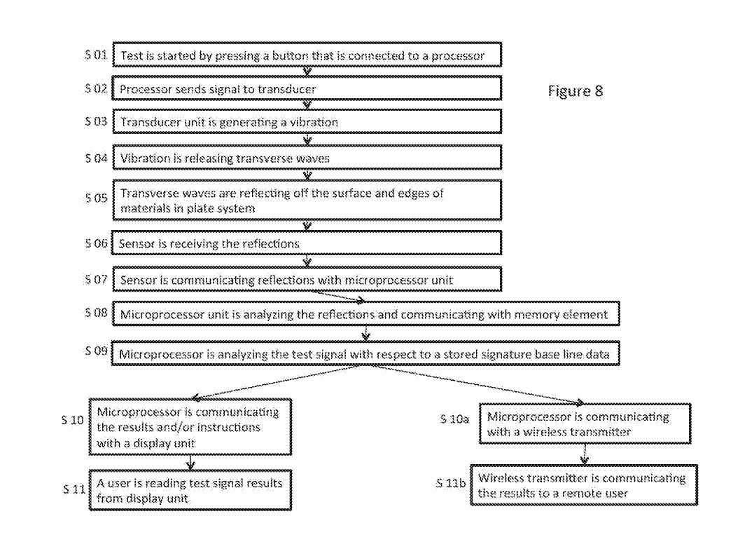

FIG. 7a illustrates that in some embodiments, a circuit for generating the injected pulse 1, receiving the reflected energy 4, 5, storing the resulting sensor waveform 3, comparing the waveform and displaying the result, can all be embedded within the armor plate such that the system becomes a completely self-contained unit. This allows the plate to be tested at any time and location. The user could test the plate by simply pushing a button and observe the indicator to be assured that the armor plate is still good.

In an alternate embodiment, the sensor would be applied to the plate in a consistent location using a test system designed for the purpose. For example, a forward operating base or remote military installation could include a test system that has a cradle-jig sized for each plate type in use. The appropriate cradle would be selected for the plate to be tested, and the plate would then be inserted in the cradle. In this case the cradle may contain the transducer and would apply and receive the mechanical pulse to the plate, removing all active components and circuitry from the armor plate. Since the plates being tested would have a known transducer location, pressure and plate size, it may be possible to use a single representative waveform signature for each plate size, without the need for a fully-custom signature that represents every single plate that is manufactured. This would allow armor plates manufactured without integrated testing to be tested using similar methods.

In an embodiment, this system can have a sensor, memory element, and a known “good” waveform representation for the plate being tested. In a particular embodiment, the system can be integrated into the soldier load carriage system (such as a fabric pouch normally used for carrying the armor plate). In another embodiment, the system could reside in a separate package that the plate is loaded into for testing prior to a mission. One skilled in the art can implement an external system that uses the systems disclosed herein comprising fundamental damage detection concepts and calculations for the injected waveform magnitude and frequency, sensor size, and waveform comparison.

In an alternate embodiment, FIG. 7b illustrates a limited amount of circuitry 9 that resides on the armor plate system. This limited circuitry would include a sensor 7 and a memory chip 3 for storing a representation of the known good waveform. The circuitry used to interface to the sensor and to read from the memory chip, would be applied by the user when testing the plate. Such an external tester could be in the form of a hand-held tester, a computer, or a larger multi-channel test system. This embodiment may be used by soldiers who are already carrying a computer system, such as a hand-held smartphone, tablet or other device capable of generating an electrical pulse, receiving an electrical pulse, performing statistical calculations, and displaying a result to the user.

In an alternate embodiment, part of the circuitry can be on the plate and the remainder of the circuitry can reside external to the plate. In an embodiment, the splitting or partitioning of the circuitry and the determination of which part of the circuitry resides on the plate and which part of circuitry resides off the plate, would be determined based on the application of the particular plate and user needs.

In an alternate embodiment, the armor test signal is generated in response to a command that is received by the system from a remote location. For example, a centralized command system may wirelessly broadcast a request to the solder-carried equipment requesting that an armor test be conducted. In an embodiment, the circuitry would automatically test the armor and either return the final result to the centralized command system, or it may return only the representative waveforms, allowing the centralized command system to perform the necessary comparison and statistical analysis to determine if the armor was damaged. Such armor test signal may also be generated by the soldier's commander, by a forward operating base, at a pre-determined time (ongoing status reporting), in response to external stimuli such as an accelerometer detecting that the plate was dropped or struck, or by any other connected system, either wired or wireless.

In an embodiment, FIG. 8 illustrates the steps involved in the overall operation of a plate system. In step 1(S01), the user starts a test by, for example, pressing a button that can be connected to a microprocessor. In step 2 (S02), the microprocessor activates the transducer unit S (03) to generate a vibration. In step 4 (S04), the vibrating transducer unit releases transverse waves. In step 5 (S05), the transverse waves travel across the plate system, reflecting off the surface and edges of the plate materials. In step 6 (S06), a sensor in the plate system receives the reflections. In step 7 (S07), the sensor communicates the reflections with a microprocessor unit. In step 8 (S08), the microprocessor unit analyzes the received reflections or test signal and communicates with the memory element. In step 9 S (09), the microprocessor unit analyzes the test signal with respect to the stored signature base line data. In step 10 (S10), the microprocessor communicates the results of the analysis to a display unit. In step 11 (S11), a user is reading the results of the analysis from the display unit. In step 10a (S10a), the microprocessor unit communicates with a wireless transmitter. In step 11b (S11b) the wireless transmitter communicates the results with a remote user or entity. In an embodiment, the remote user is able to wirelessly send instructions or messages to the plate system that can be read by a user who can read or is wearing the armor plate system.

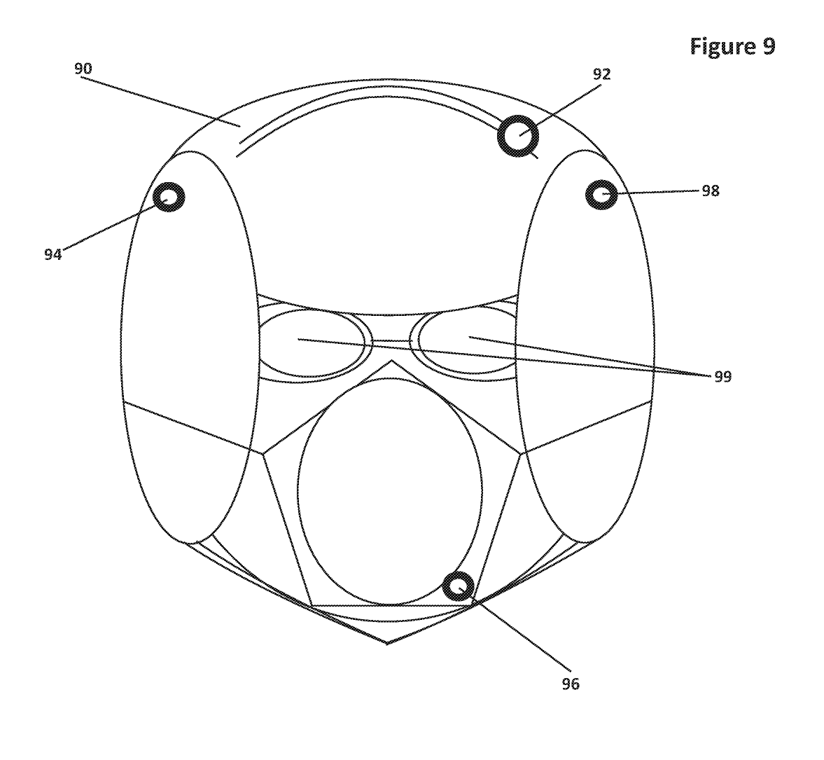

In accordance with some embodiments, FIG. 9 illustrates a damage detection system in use with various protective accessories, for example, a helmet 90, eye wear, ear coverings, and a face mask, such that the piezoelectric disk 92 is connected to the helmet, and/or embedded in the helmet such that the helmet itself becomes the plate being tested. In other embodiments, a plate with the damage detection system may be incorporated into an accessory or into apparel worn by a person. In some embodiments, there are specific sensors located on the accessories that also detect bio-parameters of the user wearing the accessories.

For example, a respiration sensor 96 may be positioned to measure the breathing rate and hydration of a wearer. In some embodiments, the flexing of a sensor detects the respiration rate. In some embodiments, the flexing of a sensor on the protective accessory tests for damage to the accessory and a pulse rate via an artery in proximity to the sensor.

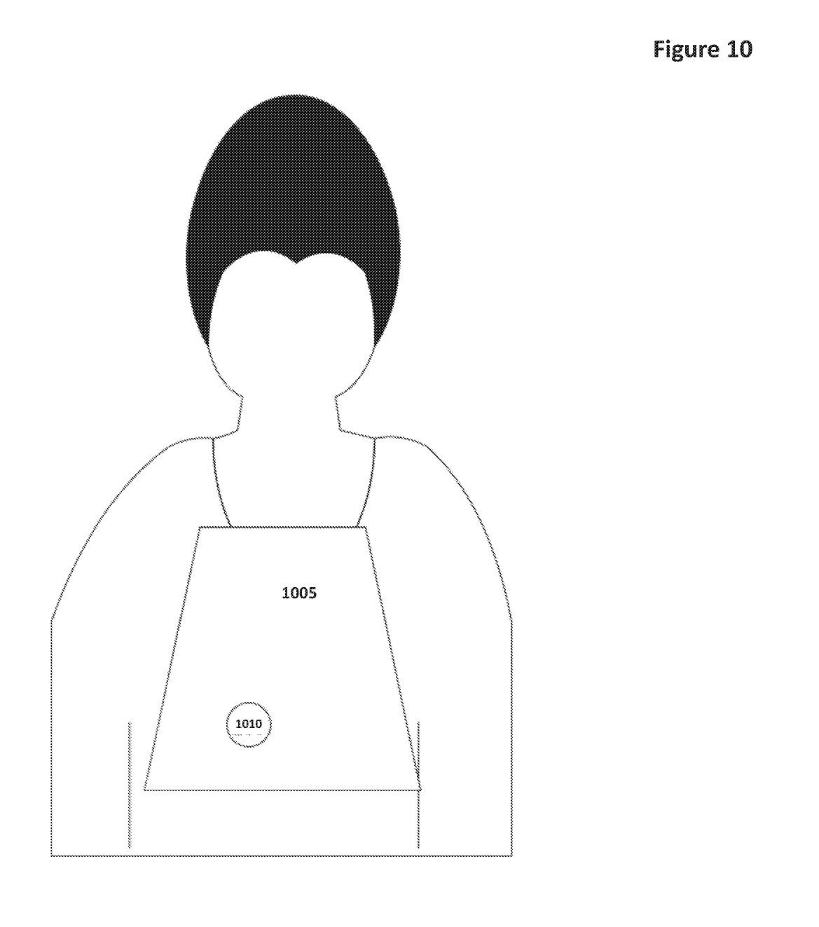

In accordance with some embodiments, FIGS. 9 and 10 illustrate a system that detects, records, processes, stores, analyzes, communicates and reevaluates the health and condition of a user who is wearing the damage detection system. In some embodiments, the health and condition of a user is determined based on bio-parameters, health signals, and other indicators of the user's health and condition. In some embodiments, vital biological signals and bio-parameters of the person are detected, recorded, processed, stored, analyzed, and communicated to the wearer or another person who may be monitoring remotely. The types of health signals and bio-parameters measured by the system are not intended to be limiting. Particular health signals can be combined in different forms depending on the intended use of the armor plate and accessory. For example, in some embodiments, measurements of some or all of respiration, heart rate, and temperature may be desired. In other embodiments, other combinations of measurements may be desired. In some embodiments, the damage detection system that detects damage in the protective apparel, armor plate, and accessories worn by the person, may be the same system that also simultaneously and/or successively detects the health and condition of a user who is wearing the damage detection system.

In accordance with some embodiments, FIGS. 9 and 10 illustrate a system including one or more sensors. The system includes a body-worn armor plate 1005 and one or more sensors 1010 that convert mechanical flex and vibration of the armor plate into an electrical signal that is representative of one or more health-related signals and conditions. In some embodiments, as illustrated in FIGS. 1a and 1b, the system incorporated on the body armor 1005 and accessory 90 comprises a memory element, a processor, a wireless transmitter, and a wireless receiver and circuitry. The system may include a user input device, whereby a user can input additional data into the system. For example, if a user is aware of a health condition tested by other means, the results and parameters of this can be inputted into the system to be incorporated into the analysis of particular signals.

FIG. 10 shows a fully assembled armor plate being worn by a human. The wearer has the armor plate 1005 mounted on his or her chest. The armor plate 1005 is curved and sized to fit a variety of body shapes and may include complex curves, thinner areas, and attachment points. These characteristics may be varied depending on the desired protection level and the particular wearer.

In some embodiments, the armor plate system is designed to be worn on the body and is generally curved and sized to fit the person wearing the armor. The armor plate is then held against the body by a vest, a pouch, a strap, or an integrated garment. Good mechanical contact of the armor plates against the body can help provide ballistic protection because the contact helps the impact force of a projectile to be absorbed by the plate and then passed onto the body in an even and repeatable way. If the armor plate is worn too loosely, the impact energy of the projectile could cause the plate to buckle, move sideways, or otherwise cause serious injury to the body.

Respiration

In some embodiments, the armor plate system detects, records, processes, stores, analyzes, communicates, can reevaluates signals and data relating to the respiration of the user wearing the system. In some embodiments, as a user of the armor plate system is breathing, the user's chest cavity and surrounding area expands and contracts, creating movement in the armor plate system. As the person begins to breathe heavily or at a faster rate, the frequency and magnitude of the expansion and contraction changes and varies. Similarly, if the user is breathing less heavily or at a slower rate, or not breathing at all, the frequency and magnitude of the expansion and contraction varies accordingly. This movement and variation is directly transferred into the armor plate which is then detected by the sensors in the system. In some embodiments, the plate in the armor flexes proportionally to the movement created by the breathing of the person wearing the system.

In some embodiments, the flexing of the plate can be measured using specifically calibrated sensors. Respiration results may be enhanced through filtering in the 0.1 Hz to 2 Hz range. In some embodiments, the sensors are piezoelectric disks that generate an electrical signal when flexed by the movement caused by the movement in the chest of the person wearing the system. Plates that contain very stiff ceramic components may not feel as if they are flexing to the subject wearing it, however, even the microscopic flexing causes by breathing can be detected with suitable sensors and signal conditioning mounted on the plate. Therefore, the plate does not have to flex in a way that is perceptible to the subject to provide this useful health-monitoring function.

In some embodiments, a processing unit analyzes the flexing vibrations received, and produces data that can be displayed or transmitted to inform the subject or base as to the condition of the subject based on the detected respiration parameters. In some embodiments, a sensor detects the rise and fall of the user's chest and records the number of respirations for one minute. One respiration may consist of one complete rise and fall of the chest, or the inhalation and exhalation of air. The processor analyzes the frequency of the respiration based on the number of rises and falls in some embodiments. In some embodiments, the processor analyzes the characteristic of the respiration in relation to the depth of breath. It is known in the art that normal respiratory rate for a healthy adult at rest is twelve to twenty breaths per minute. Such values may be stored and used by the processor to analyze a recorded respiration signal.

Heart Rate

In some embodiments, the armor plate system detects, records, processes, stores, analyzes, communicates, and reevaluates signals and data relating to the heart rate of the person wearing the armor plate. In some embodiments, the beating of the person's heart sets up vibrations and micro-flexing of the armor plate which are detected by sensors. In some embodiments, the heart rate signal is detected using the same or similar system to that employed for the respiration signal.

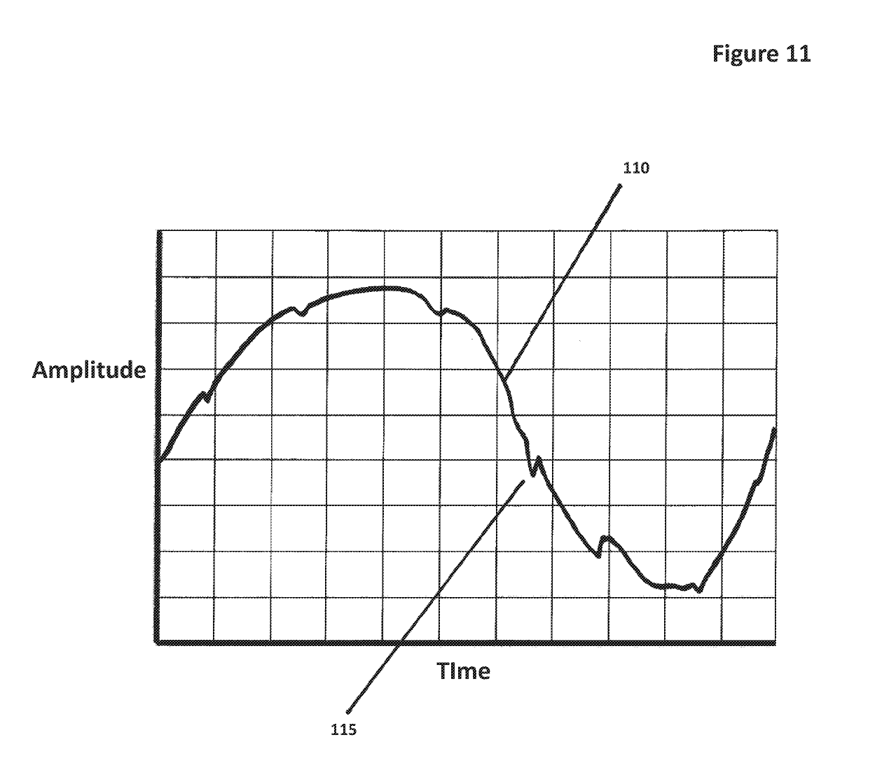

FIG. 11 is a graph showing an example of a signal 110 that may be output from a sensor mounted on an armor plate and worn on the body. Such a signal may require significant amplification, filtering, and other signal processing to remove noise and highlight the signals of interest. Respiration signals may be enhanced through filtering in the 0.1 Hz to 2 Hz range, while heart rate signals may be enhanced through filtering in the 0.5 Hz to 3 Hz range.

The signal 110 shows a large rolling signal that may be several seconds in duration. This slowly-changing signal represents the chest expansion and contraction as a person breathes. Superimposed on the signal are small deviations 115, which are caused by the heart beating.

In some embodiments, respiration and heart rate signals are detected and communicated by the same sensor. The magnitude, clarity, and location of the respiration and heart beat signals depend on the electronics and sensor type employed. In some embodiments, the heart rate signal and respiration signal are separated through filtering and signal processing techniques to convert each type of signal into two separate signal paths.

Temperature

In some embodiments, circuitry associated with the plate damage testing circuitry may be employed for additional bio-monitoring tasks. For example, if the microprocessor used to operate the armor plate damage detection test electronics includes a temperature sensor, the temperature sensor may be used to measure the temperature of the person wearing the plate. The plate worn by the user is in close contact with the user's body and this close physical contact of the plate to the body affords excellent thermal coupling between the two systems.

Generally a microcontroller would be included in the circuitry attached to the sensor. Such a microprocessor may contain a temperature sensor. A microprocessor may be built into the armor plate structure to be sufficiently close to the body to allow thermal coupling, which in turn allows the temperature of the body to be tested.

Perspiration

In some embodiments, the armor plate system has a perspiration sensor and analyzer that includes a circuit board with conductive tracks, an embedded sensor, microfluidic channels, ion-selective membranes, and/or other sensors that can detect and measure the level and composition of perspiration from the person wearing the system. In one embodiment, the perspiration sensor is integrated into the armor system in areas where the perspiration rate can be most effectively monitored depending on the desired use. Monitoring the volume and composition of perspiration can provide information on hydration, exhaustion, nutrition, and overall health. Placement of the sensor may be located on the underside of the armor close to the under arm area, the collar area, or the neck area as some examples.

Movement Capacity

In some embodiments, the user's body activity and orientation are measured through integrated vibration, acceleration, gravity, magnetic, and/or axial rotation sensors. In one embodiment, the sensors are included with the circuitry used to test the armor plate itself, or may be located separately depending on the intended use or activity. In some embodiments, where a user has sustained an injury to a particular body part, the system is able to detect the movement range of the injured body part and record and report any improvements, deteriorations, or stagnations. In some embodiments, the monitoring is continuous. In other embodiments, the monitoring is set to particular time intervals depending on the nature, location, type of injury, and/or prognosis. In some embodiments, the monitored signal is recorded and communicated to a remote location, for example a health facility. In one embodiment, the monitored signals are integrated and used as a part of an external health system that is related to the user. In some embodiments, the user's progress provides information to decision makers, locally and remotely, who may alter or maintain strategic decisions based on a user's condition.

In some embodiments, the system worn by one user is able to communicate with a system worn by one or more different users such that a network of health condition signal systems is generated. According to one embodiment, the network generates information as to the condition of a team of users, or a subset of a team as predefined in the processor. In some embodiments, a remote user is able to communicate with the system worn by a user and with the network generated by a group of systems worn by a team.

Hydration

In some embodiments, perspiration and blood pressure signals are received and analyzed by a processor to generate a qualitative indication of hydration levels.

Blood Oxygen Levels

In some embodiments, blood oxygen levels are measured using pulse oximetry. In one embodiment, light and laser technology is used to detect the level of oxygen in the blood. In some embodiments, the system takes into account factors that may affect the oxygen level reading. For example, factors such as decreased peripheral blood flow due to an injury may be taken into consideration. Light exposure, movement of test site area, anemia, abnormal warmth or coolness at the test site area, sweating at the test site area, and/or smoking may be taken into consideration by sensors and analyzed when reading the blood oxygen level.

Sensors

In some embodiments, for example, as shown in FIGS. 9 and 10, a sensor 1010, 92 is attached to the armor plate 1005 and/or to an accessory 90. The sensor is shown in the figures as a circle for ease of illustration. The particular type of sensor may depend on the type of damage detection being used. For example, the sensor may be a single piezo-electric disk that is coupled with a microcontroller. In other embodiments, there may be a series of sensors or even a series of smaller plates and sensors. The sensor may be of a type that is able to detect vibrations and flexing of the armor plate. Piezoelectric disks create electricity when they are flexed. In this manner, in various embodiments, the sensor 1010, 92, 94, 96, 98, 99 produces electrical energy when flexed, and such flexing can be caused by respiration, pulses in an artery, and heartbeats.

Sensors that operate in a similar manner to a piezoelectric disk may be used in some embodiments. Such sensors can produce an electrical representation of flex or compression of the ceramic surface. These sensor types include magnetic coils such as the voice-coil from a speaker, painted-on electroactive coatings, MEMS devices, load-cells, resistive traces, and any other suitable sensor that converts mechanical energy into an electrical signal. A piezoelectric disk may be used as it is inexpensive, light, thin and temperature stable. Other suitable sensors may be used in some embodiments.

In some embodiments, the system has one or more sensors for detecting a particular type of signal in the system. In one embodiment, the system has one or more sensors for detecting a particular range of a signal or signals in the system. In one embodiment, the system has one or more sensors for detecting a specific frequency for a particular signal. In a particular embodiment, a sensor may be configured to detect more than one signal. In a particular embodiment, a sensor may be a transistor that both generates a signal and detects that same generated signal. In another embodiment, a sensor that detects its own generated signal can also detect signals from other sensors.

According to some embodiments, the system has an armor plate damage detection system that uses a sensor to detect damage to the armor plate. In some embodiments, the same sensor is also used to detect particular health signals.

According to some embodiments, particular sensors may be embedded into the system. In other embodiments, the sensors may be attached in a way that allows a user to customize and choose which sensors to include in a particular system. The respiration rate may be sensed using a piezoelectric disk 96 and/or any other suitable sensor. The heart rate sensor may include bio-potential nodes and/or a piezoelectric disk 1010, or any other suitable sensor. The temperature sensor may include one or more of the following sensors: a thermometer, an infrared sensor, a thermistor, a thermocouple, and/or a resistance thermometer. The perspiration sensor may include one or more of: conductive tracks, moisture sensors, microfluidic channels, ion- selective membranes, and/or any other suitable sensor. Sensors for range of movement and movement capacity may include one or more of:

a motion sensor, bio-potential nodes, and any other suitable sensor. Sensors for oxygen levels and concentrations may include a pulse oximeter or any other suitable sensors. Hydration levels may be sensed using one or more of microfluidic channels, ion-selective membranes, and any other suitable sensors. Fatigue may be scanned using an iris scanner 99, bio-potential nodes, microfluidic channels, ion-selective membranes, or any other suitable sensor.

In some embodiments, signals are generated and detected to provide information relating to ambient conditions. For example, a user wearing the system may be in an environment that is humid, hot, and at a high altitude. Or conditions could be cool, and the user could be at a low altitude. The system may include sensors which detect these conditions and store the detected data on the memory element. In some embodiments, the ambient conditions are detected by the sensors, stored in the memory component, processed, and analyzed by the processor. The ambient conditions are analyzed relative to the signals detected relating user's health signals.

According to some embodiments, the system has a storage element that stores recorded signals generated by the system. The generated and detected signals that are stored on the memory component can be different types and ranges of health-related signals and biofeedback signals, including, but not limited to, respiration rate, heart rate, temperature, perspiration, range of movement, movement capacity, oxygen levels/concentration, hydration levels, fatigue, and blood pressure.

In some embodiments, the system has a wireless transmitter and receiver that can send and receive signals, data, and messages.

In some embodiments, the system has a processor or microprocessor that is programmed to receive signals from the system. The processor may be in communication with the memory element, sensors, transmitters, receivers, and other units from which data can be received and to which data can be sent. The processor may analyze the received signals by taking into account previously stored information that is located on the memory element. In some embodiments, normal or expected values for each parameter are stored. The parameters may be customized based on the user's specific health conditions. The processor may receive, process, analyze, evaluate, and deliver a generated result to the memory element. A timestamp may be recorded for each detected and recorded signal. In some embodiments, the processor initiates a particular sensor to be activated in order to receive and record subsequent values in the event that there is a value that is deemed of concern or out of normal ranges and requires further monitoring. The processor may analyze the signal detected from the user relative to and in consideration of ambient environment conditions. In some embodiments, the processor analyzes signals detected from the user taking into account any damage detected in the armor plate itself.

Data and information gathered from the sensors and the processor may be stored and/or transmitted to another location or used on location. For example, soldiers carry tablets or smart phones, and these devices may be used to display the biometric information, or they may be used to relay the information to a remote monitor, section commander, or health care professional. In some embodiments, a person monitoring the generated signals can communicate measures that need to be taken to stabilize any out-of-range values.

The use of the term armor plate is used to refer to the plates normally worn on the torso. However, protective equipment of any kind, including helmet, eyewear, groin, and extremity protection are all classed generally as personal protective equipment and can benefit from the application of integrated sensor technologies as described herein.

The various bio-monitoring systems and bio-sensors described herein may be used in combination with the armor plate damage detection systems described herein.

Although the description above contains much specificity, these should not be construed as limiting the scope of the invention but as merely providing illustrations of the various embodiments. Thus the scope of the invention should be determined by the appended claims and their legal equivalents.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

For purposes of this patent application and any patent issuing thereon, the indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

The use of “including,” “comprising,” “having,” “containing,” “involving,” and/or variations thereof herein, is meant to encompass the items listed thereafter and equivalents thereof as well as additional items.

It should also be understood that, unless clearly indicated to the contrary, in any methods claimed herein that include more than one step or act, the order of the steps or acts of the method is not necessarily limited to the order in which the steps or acts of the method are recited.

The foregoing description of various embodiments are intended merely to be illustrative thereof and that other embodiments, modifications, and equivalents are within the scope of the invention recited in the claims appended hereto.

What is claimed is:

1. A method of measuring respiration rate comprising:
(a) using a sensor on an armor plate to sense a flexing of the armor plate while the armor plate is worn by a user to produce flexing measurements; and
(b) determining at least one of a respiration rate and a heart rate of the user based on at least the flexing measurements.

2. A method as in claim 1, wherein the sensor comprises a piezoelectric sensor.

3. A method as in claim 1, wherein act (b) comprises determining a respiration rate of the user.

4. A method as in claim 1, wherein act (b) comprises determining a heart rate of the user.

5. A method as in claim 1, wherein the sensor is embedded in the armor plate.

6. A method as in claim 1, wherein act (b) comprises using a processor to determine at least one of a respiration rate and a heart rate of the user based on at least the flexing measurements.

7. A device comprising:
an armor plate;
a sensor positioned on the armor plate to measure flexing of the armor plate;
a processor configured to determine at least one of a respiration rate and a heart rate of a wearer of the armor plate, the processor using flexing data produced by the sensor.

8. A device as in claim 7, wherein the sensor comprises a piezoelectric sensor.

9. A device as in claim 7, wherein the processor is configured to determine a respiration rate of the user.

10. A device as in claim 7, wherein the processor is configured to determine a heart rate of the user.

11. A device as in claim 7, wherein the sensor is embedded in the armor plate.

12. A device comprising:
an armor plate;
a first sensor on the armor plate, the first sensor configured to measure flexing of the armor plate when the armor plate is worn by a user;
a temperature sensor on the armor plate; and
a perspiration sensor on the armor plate.

13. A device as in claim 12, further comprising bio-potential nodes to measure heart rate.

14. A device as in claim 12, wherein the first sensor comprises a piezoelectric sensor.

15. A device as in claim 12, further comprising a processor configured to determine at least one of a respiration rate and a heart rate of the user of the armor plate, the processor using flexing data produced by the sensor.

16. A device as in claim 15, wherein the processor is configured to determine the respiration rate of the user.

17. A device as in claim 15, wherein the processor is configured to determine the heart rate of the user.

18. A device as in claim 12, further comprising a motion sensor on the armor plate.
